(19) United States
(12) Patent Application Publication
(10) Pub. No.: US 2016/0346417 A1
(43) Pub. Date: Dec. 1, 2016

(54) WATERLESS URINAL CAKE COMPOSITIONS

(71) Applicant: Patrick Barden, Woodstock, GA (US)

(72) Inventor: Patrick Dennis Barden, Woodstock, GA (US)

(21) Appl. No.: 15/096,705

(22) Filed: Apr. 12, 2016

Related U.S. Application Data
(60) Provisional application No. 62/168,868, filed on May 31, 2015.

(51) Int. Cl. A61L 9/01

(52) U.S. Cl. CPC A61L 9/01

(57) ABSTRACT

Compositions capable of exuding a sealant liquid to a waterless urinal over time are described. The compositions have a base component including at least one substance that dissolves as it encounters urine, and a sealant liquid that is encapsulated by the base component and is released from the composition as the base component dissolves. The rate of dissolution of the base component may be controlled by a substance having hydrophobic and hydrophilic properties. The base component may further include other substances depending upon implementation.

### CROSS-REFERENCE TO RELATED APPLICATION DATA

This application claims priority to U.S. Provisional Patent Application Ser. No. 62/168,868, which is incorporated herein by reference in its entirety.

### FIELD OF TECHNOLOGY

The present disclosure relates generally to waterless urinal cakes, and more particularly to time-released waterless urinal cake compositions.

### BACKGROUND

Traditional urinals use water to flush urine through a curved pipe (i.e., a p-trap) each time the urinal is used. The p-trap of these urinals maintain water, thereby creating a vapor seal that prevents sewer gases from entering the restroom through the urinal. In contrast, waterless urinals have pitched piping that allows urine to flow naturally (i.e., due to gravity) to either a central discharge pipe or a main sewer line (without the need of water flushing).

Some waterless urinals use a cartridge, which is inserted into the drainage piping, to prevent sewer gases and urine odors from entering the restroom through the urinal. Other waterless urinals use non-replaceable p-traps integrated within the drainage piping. P-traps of waterless urinals use water and oil, or some other liquid that floats atop the water, to stifle sewer and urine odors.

In any event, both cartridges and p-traps of waterless urinals use a sealant liquid (e.g., oil) that floats atop of the cartridge, or water in the p-trap. Specifically, urine is able to flow through the sealant liquid while the sealant liquid acts as a vapor barrier and prevents odors from emerging back up through the drainage piping to the restroom. Urine, either through chemical reactions or physical encounters, over time, diminishes the amount of sealant liquid in the cartridge or p-trap, thereby requiring the sealant liquid be replaced by establishment personnel. Diminishment or degradation of the sealant liquid depends in part upon the frequency in which the sealant liquid encounters urine, thereby making replacement of cartridges and the sealant liquid of p-traps unique to each waterless urine.

### SUMMARY

The present disclosure generally provides compositions capable of exuding a sealant liquid to a waterless urinal over time. The composition includes a base component including one or more substances that dissolve as they encounter urine, and a sealant liquid (i.e., a substance having a density less than water) that is encapsulated by the base component and is released from the composition as the base component dissolves. The rate of dissolution of the base component may be controlled by the presence of specific additives within the composition.

Moreover, the base component may include a visual indicator that indicates when the composition should be replaced by or supplemented with additional composition. The base component may also include a fragrant material. The base component may further include other substances depending upon implementation.

### BRIEF DESCRIPTION OF THE DRAWINGS

Embodiments of devices, systems, methods, and compositions are illustrated in the figures of the accompanying drawings, which are meant to be exemplary and non-limiting, in which like references are intended to refer to like or corresponding parts, and in which:

FIG. 1 illustrates a composition for dispensing a sealant liquid to a waterless urinal over time according to the present disclosure;

FIG. 2 is a process flow diagram illustrating a method for preparing a composition according to the present disclosure;

FIG. 3A is an isometric view of an apparatus for distributing a sealant liquid to a waterless urinal over time according to the present disclosure;

FIG. 3B is a side view of an apparatus for distributing a sealant liquid to a waterless urinal over time according to the present disclosure.

### DETAILED DESCRIPTION

The detailed description of the present disclosure set forth herein makes reference to the accompanying drawings, which show various embodiments by way of illustration. While these various embodiments are described in sufficient detail to enable those skilled in the art to practice the disclosure, it should be understood that other embodiments may be realized and that logical, physical, and chemical changes may be made without departing from the spirit and scope of the disclosure. Thus, the detailed description herein is presented for purposes of illustration only and not of limitation. For example, the steps recited in any of the method or process descriptions may be executed in an order other than as presented and are not limited to the order presented. Moreover, references to a singular embodiment may include plural embodiments, and references to more than one component may include a singular embodiment.

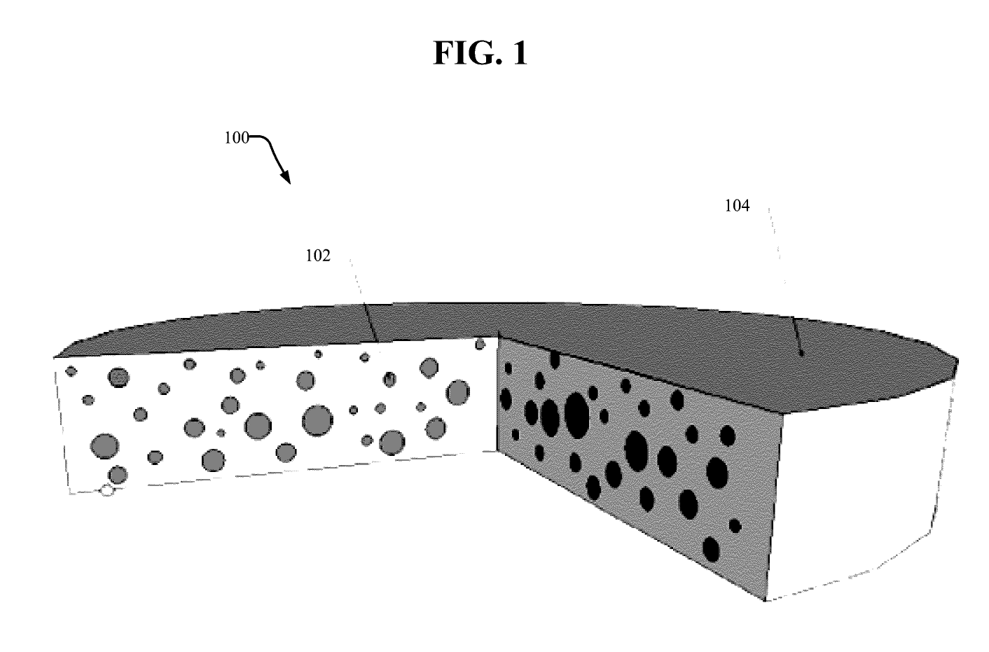

FIG. 1 illustrates a composition 100 capable of dispensing a sealant liquid to a waterless urinal over time according to the present disclosure. The composition 100 may be a hard or soft solid depending upon the makeup of the composition 100. Alternatively, the composition 100 may be a colloid (or colloidal dispersion). Yet further, the composition 100 may take the form of an amorphous, solid-liquid state gel. Depending upon the waterless urinal into which the composition 100 is placed and/or the chemical or physical makeup of the composition 100, the composition 100 may be independently placed within the waterless urinal, or may be placed within a cage/holder/housing permanently or removably implanted within the waterless urinal and/or the piping connected thereto.

It should be appreciated that the composition(s) 100 described herein may be formulated for use within a variety of waterless urinals. For example, the composition 100 may be formulated to dispense less sealant liquid 102 over a given time for waterless urinals that do not require much sealant liquid 102. Alternatively, the composition 100 may be formulated to dispense more sealant liquid 102 over a given time for waterless urinals that require more sealant liquid 102. The composition 100 may exhibit an amount of exuded liquid on its surface.

The composition 100 may be formulated to dispense a sealant liquid 102 at a rate similar or identical to a rate at which sealant liquid is lost/removed from the waterless urinal. For example, the composition 100 may be formulated to have a viscosity of about 500 poiseuille per second (cP/s or mPa/s), more preferably about 750 cP/s, and most preferably about 1000 cP/s or more as measured at about 50° C. (about 120 ° F. to about 122 ° F.). Thus, it should be appreciated that the composition 100 described herein may be specifically formulated according to the unique properties of different waterless urinals.

The composition 100 includes a sealant liquid 102 and a base component 104 that encapsulates or entraps the sealant liquid 102. The base component 104 includes at least one hydrophilic substance whose physical form, over time, changes on contact with urine. As the hydrophilic substance comes into contact with urine, the base component 104 dissolves and releases sealant liquid 102 to the waterless urinal, thereby causing a given volume of sealant liquid to be maintained or substantially maintained within the waterless urinal over time.

The sealant liquid 102 should have a density less than water (e.g., less than about 1.0 grams per milliliter (g/mL)) at about 25° C. (about 77° F.). The sealant liquid 102 should also be capable of forming a vapor barrier through which sewer and urine odors cannot pass or can only pass through in miniscule amounts. It may be beneficial for the sealant liquid 102 to have a specific gravity less than or equal to about 0.98 g/mL at about 25° C. (about 77° F.) and it may be more beneficial for the sealant liquid 102 to have a specific gravity of less than about 0.95 g/mL at about 25° C. (about 77° F.). It may also be beneficial for the sealant liquid 102 to have a water solubility less than or equal to about 0.05 grams per liter (g/L) in water at about 20° C. (about 68° F.).

It may also be beneficial for the sealant liquid 102 to have a relatively low cloud point. A cloud point is a temperature below which a liquid oil forms a cloudy appearance. Clouding may indicate a phase change of the oil. Thickened oil may indicate the liquid oil is becoming too thick for urine to pass through, thereby leading to clogging of the waterless urinal drainage pathway. To prevent clogging due to the sealant liquid 102 being too thick, a suitable sealant liquid 102 may have a cloud point of about 10° C. (about 50° F.) or less.

Other qualities of a beneficial sealant liquid 102 include a melting point less than about −4° C., a flash point above about 100° C. (about 212° F.), a water solubility of less than about 0.03 grams per liter (g/L) at about 25° C. (about 77° F.), and that the sealant liquid 102 passes Environmental Protection Agency (EPA) and Registration, Evaluation, Authorization, and Restriction of Chemicals (REACH) biodegradability and toxicity testing.

The sealant liquid 102 may be a liquid oil, such as a substance having up to four primary esters (i.e., a tetra ester), a tall oil ester, and chemical equivalents thereof. Substances having up to four primary esters include sorbitan tetraoleate, pentaerythritol trioctoate, vegetable oil, 2-octyldodecyl erucate, sorbitan monooleate, and chemical equivalents thereof. Tetra ester derivatives of pentaerythritol may also be used. The sealant liquid 102 may be comprised of a primary alcohol, such as a Geurbet alcohol having eighteen (18) to twenty-two (22) carbon atoms. For example, the primary alcohol may be 2-octyldodecanol and chemical equivalents thereof. Moreover, the sealant liquid 102 may be a mixture of at least one alcohol and an oil having up to four primary esters. The sealant liquid 102 may be present in the composition 100 in the range of about 2% by weight to about 65% by weight, preferably from about 4% by weight to about 55% by weight, more preferably from about 8% by weight to about 45% by weight, and more preferably from about 20% by weight to about 30% by weight.

In an example, sealant liquid 102 may comprise a mixture of a primary alcohol and a liquid oil (e.g., vegetable oil, tall oil, etc.). When such a mixture is used, it may be beneficial for the primary alcohol (e.g., 2-octyldodecanol) to comprise at least about 10% to about 30% of the mixture.

The base component 104 may include a compound/substance that physically binds the sealant liquid 102, thereby encapsulating the sealant liquid 102. The encapsulating substance may physically retard contact of at least one hydrophilic substance in the base component 104 with urine. The encapsulating substance may chemically react with or physically dissolve in the urine as well. As such, as the encapsulating substance interacts with urine over time, it releases sealant liquid 102 to the waterless urinal. Such an encapsulating substance may be an alkyl carboxylate salt, an alkylsulfonate salt, an alkylarylsulfonate salt, chemical equivalents thereof, and mixtures thereof, for example. The base component 104 may include at least one inorganic hydroxyl or water containing component. Moreover, the base component 104 may include a water soluble inorganic acid, such as boric acid, for example. Mixtures of these substances may be balanced to provide a buffered composition 100. The encapsulating substance (e.g., the water soluble inorganic compound) may be present in the composition 100 in the range of about 40% by weight to about 95% by weight, preferably from about 45% by weight to about 85% by weight, more preferably from about 50% by weight to about 80% by weight, and more preferably about 70% by weight to about 71% by weight.

The base component 104 may also include a compound/substance having both hydrophobic and hydrophilic properties. Such a substance may chemically bind various hydrophobic and hydrophilic substances of the base component 104. By binding to both hydrophobic and hydrophilic substances, the hydrophobic/hydrophilic substance may control the degradation/dissolution time of the substance(s) that encapsulate the sealant liquid 102. This decreased degradation/dissolution may be a result of hydrophobic portions/surfaces of the hydrophobic/hydrophilic substance physically binding to the sealant liquid 102 while hydrophilic portions/surfaces of the substance physically bind to at least one inorganic component of the base component 104 (i.e., the hydrophobic portions/surfaces repel urine away from the at least one inorganic component). For example, compounds having both hydrophobic and hydrophilic portions/surfaces generally include fatty acid soaps, alkylsulfonate soaps, and alkylarylsulfonate soaps, and more particularly include sodium stearate soaps, fatty acid carboxylate soaps, sulfonate soaps, alkyl or alkylated aromatic sulfonate soaps, and chemical equivalents thereof. The hydrophobic/hydrophilic substance may be present in the composition 100 in the range of about 0.2% by weight to about 20% by weight, preferably from about 0.5% by weight to about 16% by weight, more preferably from about 0.8% by weight to about 12% by weight, and more preferably from about 3.5% by weight to about 8% by weight.

The hydrophobic/hydrophilic substance may be supplemented with sorbitan monooleate or another non-ionic detergent. The hydrophobic/hydrophilic substance may alternatively be supplemented with one or more polyethylene glycols. For example, the polyethylene glycol may be triethylene glycol, tetraethylene glycol, or a chemical equivalent thereof. The polyethylene glycol may be present in the composition 100 in the range of about 10% by weight to about 28% by weight. In any event, a mixture of a hydrophobic/hydrophilic substance with either sorbitan monooleate or a polyethylene glycol may include about 4% by weight to about 25% by weight of the sorbitan monooleate or polyethylene glycol.

As the composition 100 of the present disclosure interacts with urine, it dissolves, degrades, and over time completely exudes its oil component and eventually has to be replaced or supplemented. As such, the composition 100 (specifically the base component 104) may optionally include a visual indicator that signals when additional composition 100 should be added to the waterless urinal. The visual indicator may be a water-soluble dye that dissolves as urine encounters the composition 100. Alternatively, the visual indicator may be a dye that does not dissolve, but instead is physically or chemically bound to a water-soluble substance of the base component 104, which disappears from the composition 100 over time when encountered by urine. Examples of visual indicators that may be incorporated within the base component 104 include methylene blue dye and chemical equivalents thereof. The visual indicator may be about 0.02% by weight to about 0.15% by weight of the composition 100.

Since the composition 100 of the present disclosure interacts with urine, it may be beneficial for the base component 104 to include a fragrant material. For example, the fragrant material may be lime oil, pine oil, citronellal aluminate, and equivalents thereof. The fragrant material may be present in the composition 100 in the range of about 0.5% by weight to about 4% by weight.

In addition, the base component 104 may include a substance that decreases the rate at which the fragrant material is released from the composition 100 (i.e., decreases the rate that urine activates the release of the fragrant material). For example, the substance may chemically or physically bind to the fragrant material and another substance of the base component 104 that is dissolved by urine slower than the fragrant material is activated by urine. Such a fragrant material activation inhibitive substances may include a hydrophobically modified, partially filled montmorillonite clay and physical or chemical equivalents thereof. The fragrant material activation inhibitive substance may compose up to about 30% by weight of the composition 100.

Instead of or in addition to using a fragrant material, a substance that possesses a trace amount of iron may be used. A trace amount of iron may inhibit the production of ammonia from urine by inhibiting certain enzymes that break down urine. For example, substances that produce trace amounts of iron upon dissolution include iron containing salts and chemical equivalents thereof. For example, in substitution of or in addition to an iron containing salt, ferric oxide may be used. The iron providing substance may be present in the composition 100 in the range of about 0.01% by weight to about 30% by weight, preferably from about 0.02% by weight to about 20% by weight, more preferably about 0.03% by weight to about 10% by weight, and more preferably from about 1.2% by weight to about 1.6% by weight. Preferably, the iron providing substance will be present in the composition 100 such that iron is present in the trapped urine in the container in a concentration of about 100 parts per million (ppm) or another concentration sufficient to inhibit such enzymatic activity.

The base component 104 may also include a preservative. For example, the preservative may be ascorbic acid and chemical equivalents thereof. The preservative acts to retard oxidation of the more sensitive components in the composition. The preservative may comprise from about 0.1% to about 1.5% of the weight of the composition 100.

Since the composition 100 may be located in an unsanitary location, it may be beneficial for the base component 104 to include an antimicrobial and/or antifungal agent. Inclusion of an antimicrobial and/or antifungal agent may decrease or slow biological attack of the sealant liquid 102 present within the composition 100. Examples of antimicrobials and antifungals include methylparaben, propylparaben, and chemical equivalents thereof. The antimicrobials and/or antifungals may be present in the composition 100 in the range of about 0.04% by weight to about 0.1% by weight, and preferably about 0.05% by weight.

The composition 100 may also include a disinfectant, such as trimethyloctadecylammonium bromide and chemical equivalents thereof. The disinfectant may comprise about 0.05% by weight to about 1% by weight of the composition 100, and preferably about 0.2% by weight of the composition 100.

The composition 100 may also include a gelatin mixture and/or a sugar mixture. The gelatin mixture may be composed of about three (3) cups of water per about six (6) tablespoons of gelatin, about three (3) cups of water per about five (5) tablespoons of gelatin, about three (3) cups of water per about four (4) tablespoons of gelatin, about three (3) cups water per about three (3) tablespoons of gelatin, about three (3) cups water per about two (2) tablespoons of gelatin, and about three (3) cups water per about two and one-half (2.5) tablespoons of gelatin. The sugar mixture may be composed of about two (2) cups of water per about one (1) cup of white sugar, about two (2) cups of water per about three-fourths (3/4) cup of white sugar, about two (2) cups of water per about one-half (½) cup of white sugar, and about two (2) cups of water per about one-fourth (¼) cup of white sugar. Some illustrative compositions 100 including gelatin mixture and/or sugar mixture include:

1. 10% sealant liquid, 90% gelatin mixture;

2. 20% sealant liquid, 80% gelatin mixture;

3. 30% sealant liquid, 70% gelatin mixture;

4. 60% boric acid, 20% vegetable oil, 10% sugar mixture, 5% soap, 5% salt;

5. 60% boric acid, 15% vegetable oil, 15% sugar mixture, 5% soap, 5% salt;

6. 56% boric acid, 15% vegetable oil, 20% sugar mixture, 5% soap, 4% salt;

7. 40% sealant liquid, 60% gelatin mixture;

8. 60% boric acid, 20% vegetable oil, 10% gelatin mixture, 5% soap, 5% salt; and

9. 60% boric acid, 15% vegetable oil, 15% gelatin mixture, 5% soap, 5% salt.

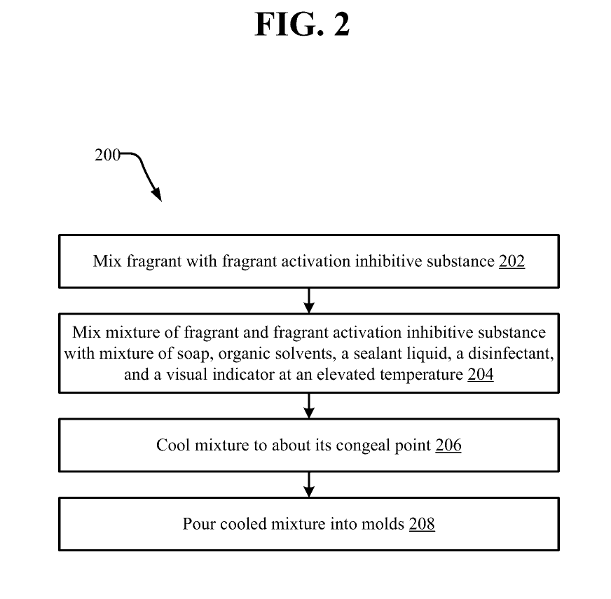

FIG. 2, illustrates a method 200 for preparing the composition 100 of the present disclosure. According to the method 200, a fragrant material is mixed with a fragrant activation inhibitive substance (illustrated as 202). The fragrant material may be pine oil and may comprise about 4% by weight of the composition 100. A portion of the fragrant activation inhibitive substance may be dry montmorillonite clay which may comprise up to about 30% by weight of the composition 100.

The mixture of the fragrant material and the fragrant activation inhibitive substance is added to a mixture of soap, organic solvents, a sealant liquid, a disinfectant, and a visual indicator at an elevated temperature (illustrated as 204). For example, the substances may be mixed at about 80° C. (about 170° F. to about 180° F.). The soap may be sodium stearate soap and may comprise about 8% by weight of the composition 100. One of the organic solvents may be tetraethylene glycol and may comprise about 10% by weight of the composition 100. Another organic solvent used may be triethylene glycol and may comprise about 28% by weight of the composition 100. The sealant liquid may be 2-octyldodecanol and may comprise about 20% by weight of the composition 100. The disinfectant may be trimethyloctadecylammonium bromide and may comprise about 0.2% by weight of the composition 100. The visual indicator may be methylene blue and may comprise about 0.002% by weight of the composition 100.

The mixture produced by step 204 is cooled to about or just above the congeal point of the mixture (illustrated as 206). The cooled mixture is then poured into molds (illustrated as 208). For example, the molds may be polypropylene lined.

### Test Parameters

Detailed below are various compositions produced and tested according to the teachings of the present disclosure. The overall testing environment in which the examples were tested was maintained at about 18° C. (about 64° F. to about 65° F.).

A slump test was performed on each of the tested compositions. A slump test is used to determine the suitability of an amorphous solid/liquid composition (i.e., the effect of gravity on the composition). A composition passes the slump test if, even though the composition feels oily to the touch, no more than one drop of oil separates at about 18° C. after 24 hours. A failure to pass the slump test is an indication that the composition may release oil originally encapsulated within the composition earlier than desired.

Water blast testing was performed to define a longevity factor (i.e., a dissolution rate) of each composition. The water blast test was performed using 150 mL of water for each blast. After the first 150 blasts at about 55° F. to about 60° F., the temperature was raised to about 70° F. to about 75° F. Each blast constituted a steady stream of water dropping about 20 cm to about 25 cm onto the target cake over a 25 second period.

As will become apparent upon a review of the below examples, some compositions included ground up boric acid crystals and 2-octyldodecanol. In contrast to being unground, ground up boric acid presents a larger surface area for entrapment of oil into microscopic crevices.

It will also be noticed that some of the tested compositions include ground up Epsom salts. By grinding up the Epsom salts, a larger surface area is generated that allows the oil to become entrapped into microscopic crevices. Furthermore, it will be noticed that some examples include ground up soap. As the soap is ground up it is mixed more thoroughly into the composition. The soap acts as a water barrier. As such, the soap, whether ground up or not, plays a role in determining the rate at which the composition disintegrates (i.e., the rate at which the water soluble inorganic ingredients will dissolve). By itself, the ground up soap dissolved over a ten (10) hour period in undisturbed water at about 18° C. (about 64° F. to about 65° F.). A slight film formed on the surface of the water.

### Tested Composition 1

A first tested composition included ground boric acid crystals, 2-octyldodecanol, ground Epsom salts, and ground soap. Table 1 below illustrates the proportionality of each component of the composition. Mixture of the components resulted in a soft soap cake.

TABLE 1
Ingredient proportionality of Test Composition 1.

| Ingredient | Approx. Weight | Approx. Weight Percentage |
|---|---|---|
| Ground boric acid | 85 g | 66.4% |
| crystals | | |
| 2-octyldodecanol | 35 g | 27.3% |
| Ground Epsom salts | 2 g | 1.6% |
| Ground soap | 6 g | 4.7% |
| Total: | 128 g | 100% |

The slump test revealed that even though the soft soap cake felt oily, it did not exude oil over a twenty-four (24) hour period. The water blast test caused several drops of sealant liquid (i.e., 2-octyldodecanol) to be released from the cake as a result of each water blast. Moreover, the water blast test demonstrated the soft soap cake began to noticeably disintegrate after five (5) water blasts, and that a portion of the soft soap cake survived for more than one hundred (100) water blasts while continuing to release all of the oil. Thus, Tested Composition 1 is an effective waterless urinal cake composition.

### Tested Composition 2

A second tested composition included ground boric acid crystals, 2-octyldodecanol, ground Epsom salts, and ground soap, similar to that of Tested Composition 1 above. However, a comparison of Tables 1 above and 2 below demonstrates the proportionality of the components of Tested Compositions 1 and 2 is different. Yet, similar to Tested Composition 1, Tested Composition 2 also resulted in a soft soap cake.

TABLE 2
Ingredient proportionality of Tested Composition 2.

| Ingredient | Approx. Weight | Approx. Weight Percentage |
|---|---|---|
| Ground boric acid | 85 g | 70.3% |
| crystals | | |
| 2-octyldodecanol | 30 g | 24.8% |
| Ground Epsom salts | 1.5 g | 1.2% |
| Ground soap | 4.5 g | 3.7% |
| Total: | 121 g | 100% |

The slump test performed on this composition revealed that even though the soft soap cake felt oily, it did not exude oil over a twenty-four (24) hour period. The water blast test caused several drops of sealant liquid (i.e., 2-octyldodecanol) to be released from the cake as a result of each water blast. Moreover, the water blast test demonstrated the soft soap cake began to noticeably disintegrate after five (5) water blasts, and that the soft soap cake survived more than eighty-five (85) water blasts while continuing to release all of the oil. Thus, while the Tested Composition 2 had a higher dissolution rate than Tested Composition 1, Tested Composition 2 is also an effective waterless urinal cake composition.

### Tested Composition 3

A further tested composition included unground boric acid crystals, vegetable oil, unground Epsom salts, and unground soap. A comparison of Tables 1, 2, and 3 demonstrates the differences between Tested Compositions 1 and 2 and Tested Composition 3 (i.e., the components of Test Composition 3 are unground and Tested Composition 3 includes vegetable oil instead of 2-octyldodecanol). Mixture of the components of Tested Composition 3 resulted in a soft solid.

TABLE 3
Ingredient proportionality of Tested Composition 3.

| Ingredient | Approx. Weight | Approx. Weight Percentage |
|---|---|---|
| Boric acid crystals | 121 g | 63.5% |
| Vegetable oil | 47 g | 25.0% |
| Epsom salts | 19 g | 10.0% |
| Soap | 3 g | 1.5% |
| Total: | 190 g | 100% |

The slump test performed on this composition revealed that even though the soft soap cake felt oily, it did not exude oil after twenty-four (24) hours. The water blast test caused many drops of sealant liquid (i.e., vegetable oil) to be released from the soft solid as a result of each water blast. Moreover, the water blast test demonstrated the soft solid began to noticeably disintegrate after two (2) water blasts, and that the soft solid survived for thirty-five (35) water blasts while continuing to release all of the oil. Thus, Tested Composition 3 has a higher dissolution rate than Tested Compositions 1 and 2. This demonstrates that tested Composition 3 is effective at supplying sealing oil at a rapid rate and is particularly effective when needed to seal a waterless urinal rapidly.

### Test Composition 4

Yet another tested composition included unground boric acid crystals, vegetable oil, and unground soap. It should be noted that the difference between Tested Compositions 3 and 4 is Tested Composition 3 includes Epsom salts whereas Tested Composition 4 does not include salt. Similar to Tested Compositions 1 and 2, mixture of the components of Tested Composition 4 also resulted in a soft soap cake.

TABLE 4
Ingredient proportionality of Tested Composition 4.

| Ingredient | Approx. Weight | Approx. Weight Percentage |
|---|---|---|
| Boric acid crystals | 141 g | 71.6% |
| Vegetable oil | 40 g | 20.3% |
| Epsom salts | 0 g | 0% |
| Soap | 16 g | 8.1% |
| Total: | 197 g | 100% |

The slump test performed on this composition revealed that even though the soft soap cake felt oily, it did not exude oil over a twenty-four (24) hour period. The water blast test caused several drops of sealant liquid (i.e., vegetable oil) to be released from the cake as a result of each water blast. Moreover, the water blast test demonstrated the soft soap cake began to noticeably disintegrate after ten (10) water blasts, and that the soft soap cake survived more than one hundred fifty (150) water blasts. Thus, Tested Composition 4 has a somewhat slower dissolution rate. Nonetheless, Tested Composition 4 may be effectively used as a waterless urinal cake, especially in urinals that do not require as rapid a replacement of sealing oil.

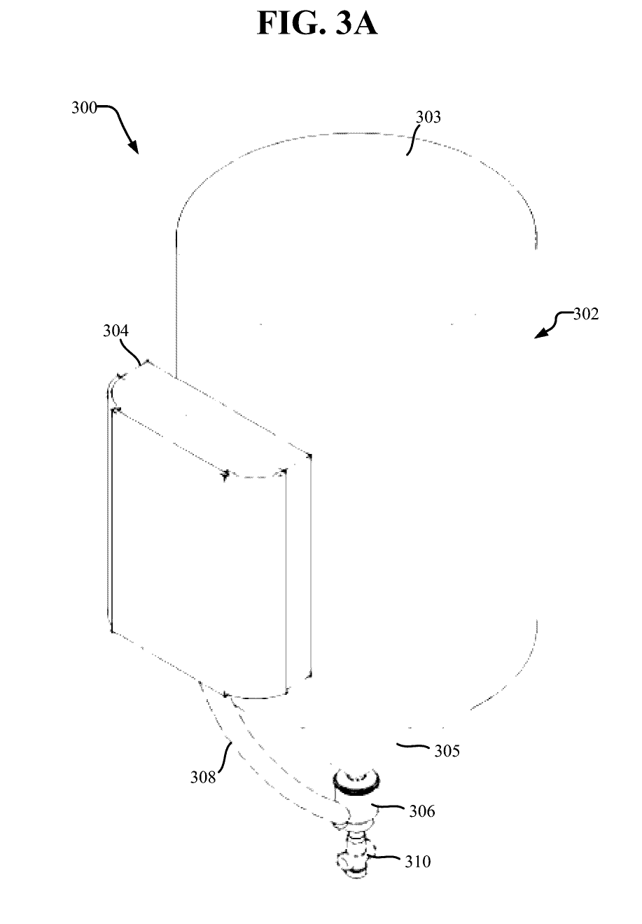

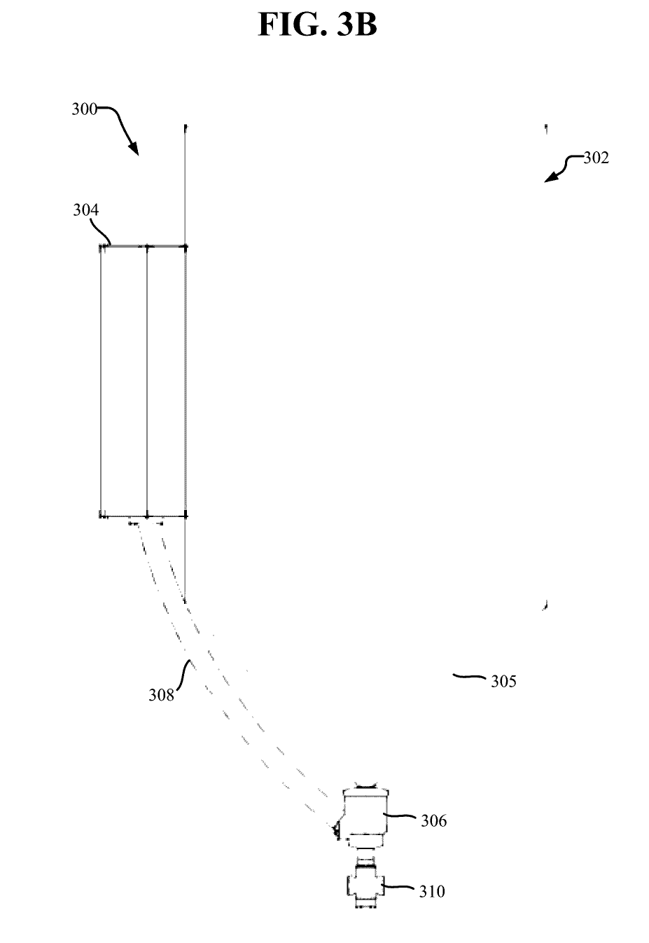

As described herein above, the composition 100 may be a hard or soft solid, a colloid (or colloidal dispersion), or an amorphous, solid-liquid state gel that is placed within a waterless urinal to provide a sealant liquid to a waterless urinal over time. Alternatively, the sealant liquid may be dispensed to the waterless urinal over time by a mechanical apparatus, such as the apparatus 300 illustrated in FIG. 3. The apparatus 300 may couple to an inner or outer surface of the waterless urinal, or to a structure (e.g., a wall) proximate to the waterless urinal, for example. One skilled in the art should appreciate that the apparatus 300 may also couple at a variety of distances away from the waterless urinal. The distance at which the apparatus 300 may be located away from the waterless urinal may depend on a length of a dispensing tube of the apparatus 300 as described herein below. For example, the apparatus 300 may be located at any distance away from the waterless urinal, provided the dispensing tube is capable of dispensing sealant liquid to the waterless urinal. Such coupling of the apparatus 300 may be permanent or temporary. For example, the apparatus 300 may be removably/temporarily coupled to the waterless urinal via a clip or other like mechanical fastener. However, one skilled in the art should appreciate that non-mechanical fasteners (e.g., epoxy, resin, and glue) may be used.

The apparatus 300 includes a main chamber 302 that stores sealant liquid to be dispensed to the waterless urinal. The main chamber 302 may have a removable portion (e.g., a lid) 303 through which sealant liquid is replenished into the main chamber 302. The main chamber 302 may be plastic, metal, or other like solid material that is inert with respect to the sealant liquid, and which is capable of storing sealant liquid over time. The main chamber 302 may be substantially tubular with a tapered portion 305 near a bottom of the main chamber 302. However, one skilled in the art should appreciate that the main chamber 302 is not so limited in shape, and may accordingly have other shapes that enable the main chamber 302 to store and dispense sealant liquid over time.

Attached to the main chamber 302 may be a power supply 304. The power supply 304 is coupled to a location of the main chamber 302 that enables the power supply 304 to control dispensation of the sealant liquid from the main chamber 302 to the waterless urinal. For example, the power supply 304 may be coupled to a side of the main chamber 302. The power supply 304 may be a wireless battery. Alternatively, the power supply 304 may be electrically coupled to a power source such as an outlet or power circuit of the establishment in which the apparatus 300 is installed.

The power supply 304 is electrically coupled to a solenoid 306 of the apparatus 300 via wiring 308. The wiring 308 may be encapsulated within a protective channel (e.g., plastic, rubber, etc.) that prevents liquids, such as urine, from contacting the wiring 308. The solenoid 306 is coupled to the main chamber 302 at a location that allows the solenoid 306 to control dispensation of sealant liquid from the main chamber 302. For example, the solenoid 306 may be coupled to a bottom portion of the main chamber 302 (as illustrated in FIG. 3). The solenoid 306 may be coupled to the main chamber 302 via a threaded connection. One skilled in the art should also appreciate that the solenoid 306 may couple to the main chamber 302 via unthreaded connections, such as via epoxy, resin, glue, friction fit, and the like, for example. The solenoid 306 includes an aperture having a diameter configured to allow sealant liquid to pass therethrough. For example, the aperture of the solenoid 306 may have a one-eighth (⅛) inch diameter.

Coupled to the solenoid 306 is a dispensing tube 310 that receives sealant liquid from the solenoid 306 and directs the sealant liquid to the waterless urinal. It should be appreciated that a length of the dispensing tube 310 is selectable based upon implementation (i.e., the length of the dispensing tube 310 may depend upon where and to what the apparatus 300 is coupled). Moreover, it should be appreciated that a circumference/diameter/cross-section (in the case of a non-circular dispensing means) of the dispensing tube 310 will depend upon implementation. For example, the circumference/diameter/cross-section of the dispensing tube 310 may be selected to exactly or substantially mate with an outlet of the solenoid 306. It should also be appreciated that the circumference/diameter/cross-section of the dispensing tube 310 and the diameter of the aperture of the solenoid 306 are selectable based upon implementation of the apparatus 300 (i.e., based upon the amount of sealant liquid desired to be released to the waterless urinal during a determined amount of time).

In operation, the power supply 304 is configured to actuate the solenoid 306 at intervals, thereby releasing sealant liquid at intervals. The solenoid 306 is actuated to maintain a desired amount of sealant liquid within the waterless urinal. Therefore, actuation of the solenoid 306 may be based on urinal usage. For example, the more a urinal is used, the more frequent the solenoid 306 may be actuated, and vice versa.

The intervals may be evenly or unevenly spaced in time. Each actuation of the solenoid 306 may release an identical or substantially identical amount of sealant liquid. Alternatively, some of the or each actuation may release a different amount of sealant liquid. The amount of sealant liquid released may be determined according to volume. A user may manually configure operation of the power supply 304 to control actuation of the solenoid 306. Alternatively, a computing device (electrically connected to the power supply 304) may be configured to automatically control the power supply 304, and accordingly the solenoid 306.

Although the present disclosure has been described herein with reference to the accompanying drawings, it is to be understood that the present disclosure is not limited to those precise teachings, and that various other changes and modifications may be made by one skilled in the art without departing from the spirit and scope of the present disclosure.

What is claimed is:

1. A waterless urinal cake, comprising:
a water soluble inorganic compound;
a sealant liquid;
a salt; and
a material that forms a barrier between hydrophobic and hydrophilic substances.

2. The waterless urinal cake of claim 1, wherein the water soluble inorganic compound is boric acid.

3. The waterless urinal cake of claim 1, wherein the sealant liquid is selected from the group consisting of a liquid oil and a primary alcohol.

4. The waterless urinal cake of claim 3, wherein the liquid oil is an oil comprising up to four primary esters and the primary alcohol is 2-octyldodecanol.

5. The waterless urinal cake of claim 1, wherein the barrier forming material is selected from the group consisting of sodium stearate soap, fatty acid carboxylate soap, alkylsulfonate soap, and alkylarylsulfonate soap.

6. The waterless urinal cake of claim 1, further comprising at least one of a fragrant material, a preservative, a visual indicator, an antimicrobial agent, or an antifungal agent.

7. A composition that releases a sealant liquid to a waterless urinal over time, comprising:
a water soluble inorganic compound;
at least one of a primary alcohol or a liquid oil; and
a soap.

8. The composition of claim 7, wherein the water soluble inorganic compound is boric acid.

9. The composition of claim 7, wherein:
the primary alcohol has between 18 and 22 carbon atoms; and
the liquid oil is selected from the group consisting of a tetra primary ester and tall oil.

10. The composition of claim 7, wherein the water soluble inorganic compound is about 50% by weight to about 80% by weight of the composition.

11. The composition of claim 7, wherein the at least one of primary alcohol or a liquid oil is about 8% by weight to about 45% by weight of the composition.

12. The composition of claim 7, wherein the soap is about 0.8% by weight to about 12% by weight of the composition.

13. The composition of claim 7, further comprising at least one of a fragrant material or an iron containing substance, wherein:
the fragrant material is selected from the group consisting of lime oil, pine oil, and citronellal aluminate; and
the iron containing substance is one of an iron containing salt or ferric oxide.

14. The composition of claim 13, wherein:
the fragrant material is about 0.5% by weight to about 4% by weight of the composition; and
the iron producing substance is about 0.3% by weight to about 10% by weight of the composition.

15. The composition of claim 7, further comprising a preservative.

16. The composition of claim 15, wherein the preservative is ascorbic acid.

17. The composition of claim 15, wherein the preservative is about 0.1% by weight to about 1.5% by weight of the composition.

18. A composition for use within a waterless urinal, comprising:
a sealant liquid that forms a vapor barrier between gases and a waterless urinal, the sealant liquid have a density less than water; and
a base component that encapsulates the sealant liquid, the base component dissolving when contacted with urine, the base component releasing sealant liquid to the waterless urinal over time as the base component is dissolved by urine.

19. The composition of claim 18, wherein:
the sealant liquid is selected from the group consisting of vegetable oil, 2-octyldodecanol, and combinations thereof; and
the base component is a water-soluble inorganic compound.

20. The composition of claim 18, further comprising a substance possessing both hydrophobic and hydrophilic properties that decreases a rate at which the base component is dissolved by urine.
